(19) United States
(12) Patent Application Publication
(10) Pub. No.: US 2023/0390217 A1
(43) Pub. Date: Dec. 7, 2023

(54) SNAP-FIT LOCKING MECHANISM FOR A WATER FILTER CARTRIDGE COVER IN A HOUSEHOLD APPLIANCE, METHOD FOR LOCKING THE CARTRIDGE COVER AND HOUSEHOLD APPLIANCE

(71) Applicant: BSH Hausgeräte GmbH, München (DE)

(72) Inventors: Narendra Mal, Knoxville, TN (US); Erhan Kaya, Tekirdag (TR); Cemalettin Hasturk, Knoxville, TN (US)

(21) Appl. No.: 18/451,160

(22) Filed: Aug. 17, 2023

Related U.S. Application Data
(63) Continuation of application No. 17/667,699, filed on Feb. 9, 2022, now abandoned.
Application No. 17/667,699 is a division of application No. 16/608,116, filed on Oct. 24, 2019, now Pat. No. 11,278,505.
(60) Provisional application No. 62/489,191, filed on Apr. 24, 2017.

(30) Foreign Application Priority Data
Sep. 13, 2022 (TR) 2022/014149

(51) Int. Cl. A61K 31/137; A61P 17/00

(52) U.S. Cl. CPC A61K 31/137; A61P 17/00; A61K 45/06

(57) ABSTRACT

A snap-fit locking mechanism assembly for a water filter cartridge cover in a household appliance includes a housing of the water filter cartridge cover being coupled to the household appliance. The housing can be positioned adjacent to a water filter cartridge within the household appliance. The water filter cartridge cover can include a cover panel positioned within the housing. The water filter cartridge cover can include an annular snap-fit lock that can rotate the cover panel between an open position and a closed position. The open position can provide access to the water filter cartridge. The water filter cartridge cover can include a cantilever snap-fit lock that can couple the cover panel to the housing in the closed position. A method for locking the cartridge cover and a household appliance are also provided.

### CROSS-REFERENCE TO RELATED APPLICATION

This application claims the priority, under 35 U.S.C. § 119, of Turkish Patent Application TR 2022/014149, filed Sep. 13, 2022; the prior application is herewith incorporated by reference in its entirety.

### FIELD AND BACKGROUND OF THE INVENTION

The present disclosure relates to household appliances. More specifically, but not by of limitation, this disclosure relates to refrigeration appliances.

Household appliances such as refrigerators can include water filter assemblies that can treat incoming water before dispensing the water to a built-in water dispenser or an automatic ice maker. The water filter assembly can include a manifold and a water filter cartridge for filtering the water. Over time, water filter cartridges may become less effective at filtering water and may require periodic replacement. In some cases, the water filter cartridge can be uncovered and can be visible and accessible for replacement.

### SUMMARY OF THE INVENTION

It is accordingly an object of the invention to provide a snap-fit locking mechanism for a water filter cartridge cover in a household appliance, a method for locking the cartridge cover and a household appliance, which overcome the hereinafore-mentioned disadvantages of the heretofore-known mechanisms, methods and appliances of this general type and which facilitate replacement of water filter cartridges.

Certain aspects and features of this disclosure relate to a household appliance that includes a water filter cartridge cover with a cantilever snap-fit lock and an annular snap-fit lock. In some examples, the household appliance can be a refrigeration appliance. The water filter cartridge cover can be moved between an open position and a closed position in front of a water filter cartridge cover. For example, the water filter cartridge cover can rotate about the annular snap-fit lock into the open position to allow access to the water filter cartridge. The water filter cartridge can then be removed and replaced. The water filter cartridge cover can also rotate about the annular snap-fit lock into a closed position that can cover and prevent access to the water filter cartridge. Additionally, the water filter cartridge cover can be locked via the cantilever snap-fit lock to lock the water filter cartridge while in the closed position.

Providing a water filter cartridge cover can allow users of the household appliance to easily and safely access the water filter cartridge without risk of exposure to other electrical components within the household appliance. In some examples, the household appliance may include a ventilation grill along a bottom portion of the household appliance. Conventionally, the ventilation grill may not cover the water filter cartridge, as doing so can prevent access to the water filter cartridge. A front-facing side of the water filter cartridge cover may include a ventilation grill that may be the same as or similar in appearance to the conventional ventilation grill. In this way, the household appliance may appear to have a seamless, uninterrupted ventilation grill along the bottom portion when the water filter cartridge cover is covering the water filter cartridge in the closed position. Since the water filter cartridge cover is locked via the cantilever snap-fit lock, the water filter cartridge cover may protect the water filter cartridge while in the closed position.

In one particular example, the water filter cartridge cover can include a cover panel that can rotate within a housing about an annular snap-fit lock. For example, the annular snap-fit lock may be positioned within a top portion of the water filter cartridge cover. The cover panel can thus rotate in an upward direction to an open position, which may allow access to a water filter cartridge behind the cover panel. The cover panel can be rotated in a downward direction to a closed position. The cover panel can be locked into the housing via a cantilever snap-fit lock positioned at a bottom portion of the water filter cartridge cover. The cantilever snap-fit lock can include a first arm on the cover panel that can mate with a second arm on the housing. The cover panel can be locked with a pushing force towards the cover panel to mate the first arm with the second arm. The cover panel can be unlocked with a pulling force in a direction away from the cover panel. The pulling force can exceed a locking force of the mated first arm and second arm, which can cause the first arm to un-mate with the second arm. The unlocked cover panel can then rotate freely about the annular snap-fit to move into the open position.

Alternatively, the annular snap-fit lock may be positioned at a bottom portion of the water filter cartridge cover. In such examples, the cover panel may rotate in a downward direction about the annular snap-fit to move into the open position. And, the cover panel may rotate in an upward direction about the annular snap-fit to move into the closed position. The cover panel may lock to the cantilever snap-fit lock at a top portion of the water filter cartridge cover.

Other features which are considered as characteristic for the invention are set forth in the appended claims.

Although the invention is illustrated and described herein as embodied in a snap-fit locking mechanism for a water filter cartridge cover in a household appliance, a method for locking the cartridge cover and a household appliance, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a diagrammatic, front-elevational view of an example of a refrigeration appliance including an uncovered water filter cartridge according to some aspects of the present disclosure;

FIG. 2 is a diagrammatic, front-elevational view of an example of a refrigeration appliance including a water filter cartridge cover according to some aspects of the present disclosure;

FIG. 3 is a diagrammatic, perspective view of an example of a water filter cartridge cover in a closed position according to some aspects of the present disclosure;

FIG. 4 is a diagrammatic, perspective view of the water filter cartridge cover of FIG. 3 in an open position according to some aspects of the present disclosure;

FIG. 5 is a diagrammatic, side-elevational view of the water filter cartridge cover of FIG. 3 in an open position according to some aspects of the present disclosure; and

FIG. 6 is a diagrammatic, side-elevational view of the water filter cartridge cover of FIG. 5 in a closed position according to some aspects of the present disclosure.

### DETAILED DESCRIPTION OF THE INVENTION

Illustrative examples are given to introduce the reader to the general subject matter discussed herein and are not intended to limit the scope of the disclosed concepts. The following sections describe various additional features and examples with reference to the drawings in which like numerals indicate like elements, and directional descriptions are used to describe the illustrative aspects, but, like the illustrative aspects, should not be used to limit the present disclosure.

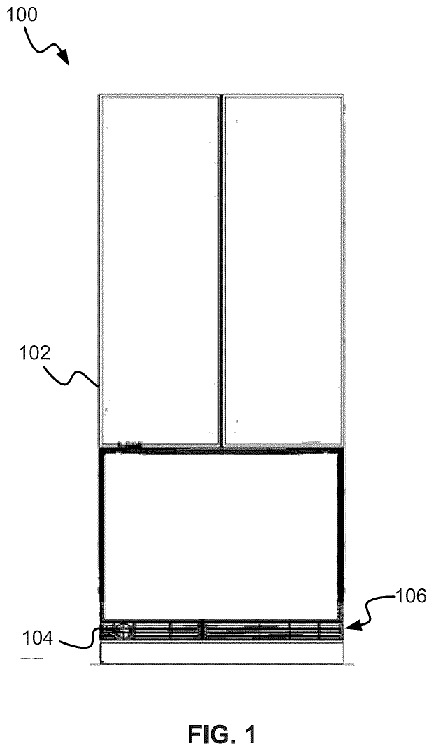

Referring now to the figures of the drawings in detail and first, particularly, to FIG. 1 thereof, there is seen a diagrammatic, front-elevational view of an example of a refrigeration appliance 100 including an uncovered water filter cartridge 104 according to some aspects of the present disclosure. The refrigeration appliance 100 can be an example of a conventional household appliance that includes an uncovered water filter cartridge 104 without a cover. The refrigeration appliance 100 can include a body 102 and an uncovered water filter cartridge 104 that is positioned adjacent to a ventilation grill 106 that runs horizontally along a bottom portion of the refrigeration appliance 100. In some examples, the uncovered water filter cartridge 104 can be positioned at other locations in the refrigeration appliance 100 or positioned vertically at a different angle relative to an axis of the refrigeration appliance 100. Because the uncovered water filter cartridge 104 is uncovered, it can be visible to a user of the refrigeration appliance 100 and may be unprotected from damage.

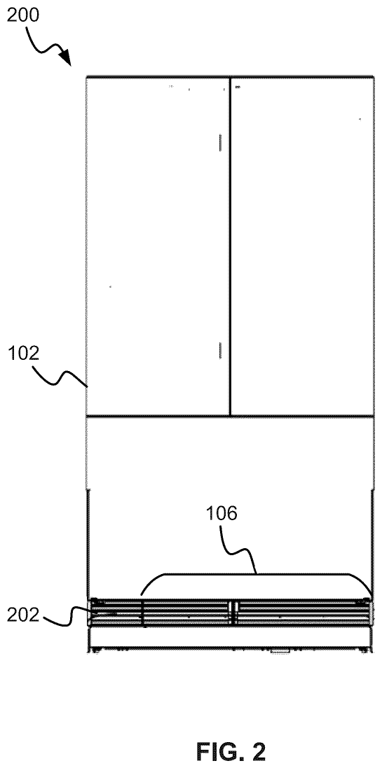

FIG. 2 is a diagrammatic, front-elevational view of an example of a household appliance 200 including a water filter cartridge cover 202 according to some aspects of the present disclosure. Although a household appliance 200 is depicted, any other household appliance may be used, such as a washing machine, a dishwasher, or any household appliance that can include a water filter assembly. The household appliance 200 can include a body 102 that can include a machine room compartment (not pictured). The machine room compartment can include compressors, a condenser fan, inverters, and a water filter assembly. The water filter assembly can be used to supply water to various components of the household appliance 200, such as an ice maker or a water dispenser. The water filter assembly can include a water filter cartridge, such as the uncovered water filter cartridge 104, which can filter water for the refrigeration appliance 100. The body 102 of the household appliance 200 may include a ventilation grill 106 that can run horizontally along a bottom part of the household appliance 200. The ventilation grill 106 may not run along an entire length of the bottom part of the household appliance 200. For example, a portion of the bottom part adjacent to the ventilation grill 106 can include the water filter cartridge cover 202.

The water filter cartridge cover 202 may cover the uncovered water filter cartridge 104 of the water filter assembly. The water filter cartridge cover 202 may appear to have fins that mimic the appearance of the ventilation grill 106. Thus, the household appliance 200 may have a seamless appearance between the ventilation grill 106 and the water filter cartridge cover 202 when the water filter cartridge cover 202 is in a closed position (e.g., covering the uncovered water filter cartridge 104). The water filter cartridge cover 202 may lock in the closed position and may protect the uncovered water filter cartridge 104 from external damage, such as from children or pets. The water filter cartridge cover 202 may unlock as a user pulls on a top or a bottom edge of the water filter cartridge cover 202. For example, the water filter cartridge cover 202 can be oriented to unlock and move to an open position (e.g., allowing access to the uncovered water filter cartridge 104) in a downward direction or an upward direction.

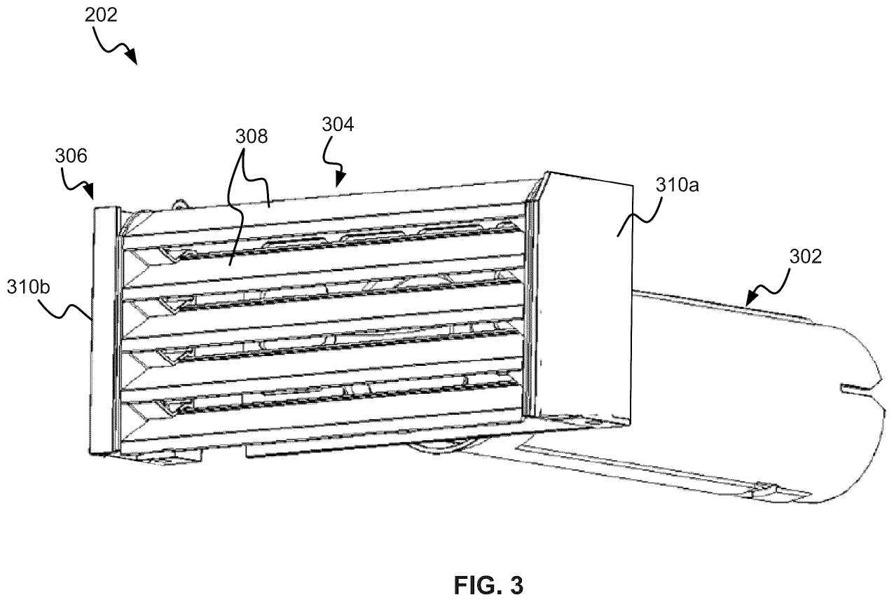

FIG. 3 is a diagrammatic, perspective view of an example of the water filter cartridge cover 202 in a closed position according to some aspects of the present disclosure. The water filter cartridge cover 202 can be positioned in front of a water filter cartridge, such as uncovered water filter cartridge 104. In the closed position, the water filter cartridge cover 202 can hide the water filter cartridge and can prevent access to the water filter cartridge. The water filter cartridge can be coupled to a manifold 302. The water filter cartridge cover 202 can include a cover panel 304 and a housing 306. In some examples, the cover panel 304 can appear as a ventilation grill, and may include multiple angled fins 308. The appearance of the cover panel 304 can match an appearance of a ventilation grill on a refrigeration appliance, such as the ventilation grill 106 on the household appliance 200. The housing 306 may be a structure that can house the cover panel 304, and may include a first end 310a and a second end 310b. The cover panel 304 can be mounted within the housing 306, such as between the first end 310a and the second end 310b. The cover panel 304 may move within the housing 306 from the closed position depicted in FIG. 3 to an open position, or vice versa.

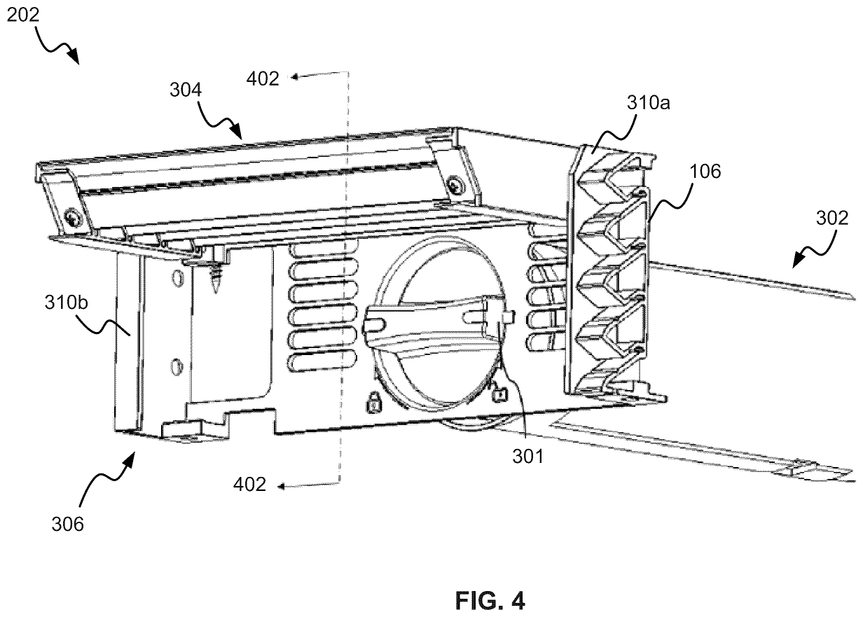

FIG. 4 is a diagrammatic, perspective view of the water filter cartridge cover 202 of FIG. 3 in an open position according to some aspects of the present disclosure. The water filter cartridge cover 202 can move from the closed position depicted in FIG. 3 to the open position depicted in FIG. 4 by rotating about an annular snap-fit lock (not depicted) that can mount the cover panel 304 to the housing 306. For example, as depicted in FIG. 4, a top portion of the cover panel 304 can be mounted by the annular snap-fit lock to the first end 310a and the second end 310b of the housing 306. The annular snap-fit lock can run from the first end 310a through the cover panel 304 to the second end 310b. The cover panel 304 can therefore rotate in an upward direction about the annular snap-fit lock from the closed position to the open position. In other examples, a bottom portion of the cover panel 304 can be mounted to the housing 306, and the cover panel 304 can rotate in a downward direction from the closed position to the open position. The housing 306 may be coupled to the ventilation grill 106 that runs along the bottom portion of the household appliance 200. For example, the first end 310a of the housing 306 can be coupled to a side of the ventilation grill 106.

By moving the cover panel 304 to the open position, a water filter cartridge 301 can be visible and accessible to a user. The user may repair, remove, or replace the water filter cartridge 301 when the cover panel 304 is in the open position. For example, the user may rotate the cover panel 304 to the open position, remove the water filter cartridge 301 (such as by rotating the water filter cartridge 301 anticlockwise or counterclockwise and pulling the water filter cartridge 301 out of the manifold 302), installing a new water filter cartridge, and moving the cover panel 304 to the closed position. In some examples, the cover panel 304 may be held by the user in the open position. When the user stops holding the cover panel 304 in the open position, the cover panel 304 may automatically rotate to the closed position.

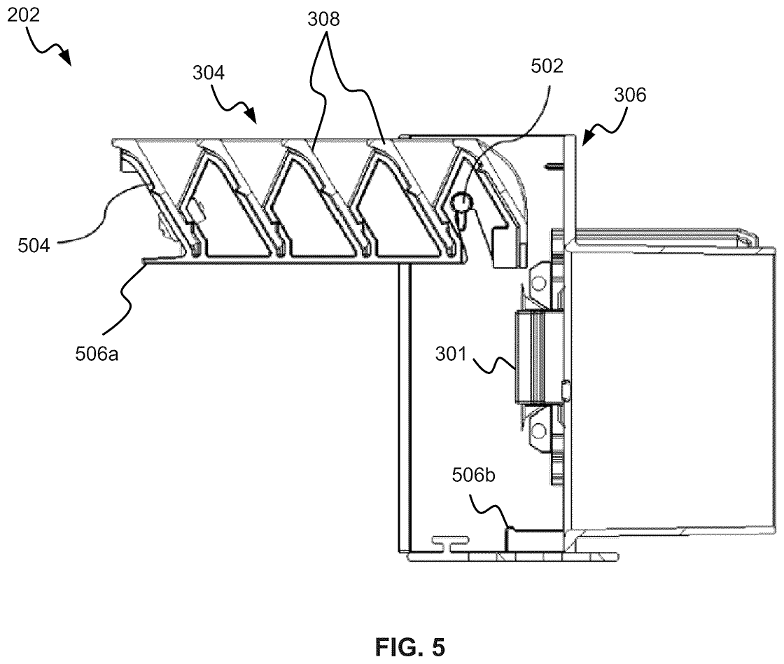

FIG. 5 is a diagrammatic, side-elevational view of the water filter cartridge cover 202 of FIG. 3 in an open position according to some aspects of the present disclosure. The diagrammatic side view is taken along a line 402 depicted in FIG. 4. The water filter cartridge cover 202 can include an annular snap-fit lock 502 that can mount the cover panel 304 to the housing 306. The cover panel 304 can rotate about the annular snap-fit lock 502 to move between the open position and the closed position. The cover panel 304 can rotate in an upwards direction into the open position, and in a downward direction into the closed position. Although the annular snap-fit lock 502 is depicted along a top portion of the water filter cartridge cover 202, in some examples the annular snap-fit lock 502 may be positioned along a bottom portion of the housing 306. In such examples, the cover panel 304 can rotate in a downward direction into the open position, and in an upward direction into the closed position.

The cover panel 304 can include a first ventilation grill that can include multiple angled fins 308. The first ventilation grill can be the same as or similar in appearance to the ventilation grill 106 depicted in FIG. 2. The angled fins 308 can be coupled to a support bracket 504 that is coupled to the housing 306 via the annular snap-fit lock 502. Portions of the housing 306 and the support bracket 504 can together form a cantilever snap-fit lock 506 that can lock the cover panel 304 to the housing 306 while in the closed position. For example, the support bracket 504 can include a first arm 506a that protrudes from the support bracket 504. The housing 306 can include a second arm 506b that protrudes from the housing 306. When the cover panel 304 is moved into the closed position, the first arm 506a can mate with the second arm 506b to lock the cover panel 304 to the housing 306.

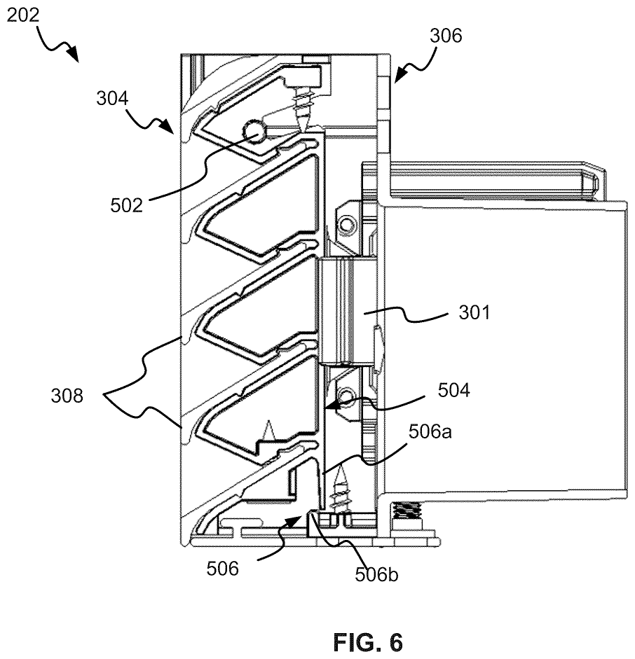

FIG. 6 is a diagrammatic, side-elevational view of the water filter cartridge cover 202 of FIG. 5 in a closed position according to some aspects of the present disclosure. In the closed position, the first arm 506a of the cantilever snap-fit lock 506 can be mated with the second arm 506b to lock the cover panel 304 to the housing 306. The second arm 506b may prevent the first arm 506a from moving past the second arm 506b until a pulling force that is greater than the mating force of the cantilever snap-fit lock 506 is exerted on the cover panel 304 to un-mate the first arm 506a from the second arm 506b. After unlocking the cantilever snap-fit lock 506, the cover panel 304 can be rotated about the annular snap-fit lock 502 into the open position.

In some examples, the cover panel 304 can automatically lock to the housing 306 via the cantilever snap-fit lock 506 in response to a user no longer holding the cover panel 304 in the open position. In other examples, the cover panel 304 may lock to the housing 306 via the cantilever snap-fit lock 506 in response to the user pushing the cover panel 304 towards the housing 306.

The foregoing description of certain examples, including illustrated examples, has been presented only for the purpose of illustration and description and is not intended to be exhaustive or to limit the disclosure to the precise forms disclosed. Numerous modifications, adaptations, combinations, and uses thereof are possible without departing from the scope of the disclosure.

The following is a summary list of reference numerals and the corresponding structure used in the above description of the invention.

### REFERENCE LIST

100. Refrigeration appliance
102. Body
104. Uncovered water filter cartridge
106. Ventilation grill
200. Household appliance
202. Water filter cartridge cover
301. Water filter cartridge
302. Manifold
304. Cover panel
306. Housing
308. Angled fins
310a. First end
310b. Second end
402. Line
502. Annular snap-fit lock
504. Support bracket
506. Cantilever snap-fit lock
506a. First arm
506b. Second arm

1. A water filter cartridge cover, comprising:
a housing coupled to a household appliance, said housing being positioned adjacent to a water filter cartridge within the household appliance;
a cover panel positioned within said housing;
an annular snap-fit lock configured to rotate said cover panel between an open position and a closed position, said open position providing access to the water filter cartridge; and
a cantilever snap-fit lock configured to couple said cover panel to the housing in said closed position.

2. The water filter cartridge cover according to claim 1, wherein said cover panel includes:
a first ventilation grill; and
a support bracket coupled to said first ventilation grill, said support bracket being coupled to said housing by said annular snap-fit lock.

3. The water filter cartridge cover according to claim 2, wherein the cantilever snap-fit lock includes:
a first arm protruding from said support bracket; and
a second arm protruding from said housing;
said first arm configured to mate with said second arm to lock the water filter cartridge cover in said closed position.

4. The water filter cartridge cover according to claim 3, wherein said cantilever snap-fit lock is configured to unmate said first arm from said second arm to unlock the water filter cartridge cover.

5. The water filter cartridge cover according to claim 2, wherein the household appliance includes a second ventilation grill, and said housing is coupled to the second ventilation grill by said annular snap-fit lock.

6. The water filter cartridge cover according to claim 1, which further comprises:
a top portion and a bottom portion of the water filter cartridge cover;
said annular snap-fit lock being positioned at said top portion and said cantilever snap-fit lock being positioned at said bottom portion; and
said cover panel being configured to rotate about said annular snap-fit lock in an upward direction into said open position.

7. The water filter cartridge cover according to claim 1, which further comprises:
a top portion and a bottom portion of the water filter cartridge cover;
said annular snap-fit lock being positioned at said bottom portion and said cantilever snap-fit lock being positioned at said top portion; and
said cover panel being configured to rotate about said annular snap-fit lock in a downward direction into said open position.

8. A method, comprising:
providing a water filter cartridge cover being coupled to a household appliance and positioned adjacent to a water filter cartridge in the household appliance;
rotating the water filter cartridge cover about an annular snap-fit lock into an open position, the open position providing access to the water filter cartridge;
rotating the water filter cartridge cover about the annular snap-fit lock into a closed position, the closed position preventing access to the water filter cartridge; and
using a cantilever snap-fit lock to lock the water filter cartridge cover to the household appliance.

9. The method according to claim 8, which further comprises:
providing the water filter cartridge cover with a housing and a cover panel including a ventilation grill coupled to a support bracket; and
carrying out the step of using the cantilever snap-fit lock to lock the water filter cartridge cover to the household appliance by:
pushing the cover panel against the housing of the water filter cartridge cover; and
mating a first arm protruding from the support bracket to a second arm protruding from the housing.

10. The method according to claim 9, which further comprises using the cantilever snap-fit lock to unlock the water filter cartridge cover by pulling the cover panel away from the housing to separate the first arm from the second arm.

11. The method according to claim 9, which further comprises using the annular snap-fit lock to couple the housing to a ventilation grill of the household appliance.

12. The method according to claim 8, which further comprises:
positioning the annular snap-fit lock at a top portion of the water filter cartridge cover and positioning the cantilever snap-fit lock at a bottom portion of the water filter cartridge cover; and
carrying out the step of rotating the water filter cartridge cover about the annular snap-fit lock into the open position by:
rotating the water filter cartridge cover about the annular snap-fit lock in an upward direction into the open position.

13. The method according to claim 8, which further comprises positioning the annular snap-fit lock at a bottom portion of the water filter cartridge cover and positioning the cantilever snap-fit lock at a top portion of the water filter cartridge cover.

14. The method according to claim 13, which further comprises carrying out the step of rotating the water filter cartridge cover about the annular snap-fit lock into the open position by:
rotating the water filter cartridge cover about the annular snap-fit lock in a downward direction into the open position.

15. A household appliance, comprising:
a body including a water filter cartridge; and
a water filter cartridge cover according to claim 1 for covering said water filter cartridge;
said water filter cartridge cover being configured to be coupled to said body and configured to rotate about said annular snap-fit lock between said open position for allowing access to said water filter cartridge and said closed position for preventing access to said water filter cartridge; and
said water filter cartridge cover being configured to be locked to said body by said cantilever snap-fit lock.
